(19) United States
(12) Patent Application Publication
(10) Pub. No.: US 2020/0281767 A1
(43) Pub. Date: Sep. 10, 2020

(54) OCULAR AGENT DELIVERY DEVICE, OCULAR MONITORING DEVICE AND METHODS THEREOF

(71) Applicant: Paloza LLC, Los Altos, CA (US)

(72) Inventor: Peter C. ZHU, Los Altos, CA (US)

(73) Assignee: Paloza LLC, Los Altos, CA (US)

(21) Appl. No.: 16/814,922

(22) Filed: Mar. 10, 2020

Related U.S. Application Data
(60) Provisional application No. 62/816,136, filed on Mar. 10, 2019.

(51) Int. Cl. A61F 9/00; A61K 9/00

(52) U.S. Cl. CPC A61F 9/0017; A61K 9/0051

(57) ABSTRACT

The present invention is directed to novel ocular drug or agent delivery devices, systems and methods, and ocular monitoring devices, systems and methods. More specifically, the present invention is directed to an ocular agent delivery device, an ocular sampling or monitoring device, and methods thereof utilizing contact lens technology in which lenses or lens assemblies are configured to contain drugs or agents that are delivered by applying to the eye in order to relieve symptoms due to conditions, such as infections and relieve eye improve comfort.

### CROSS-REFERENCE TO RELATED APPLICATION

The present application claims the priority benefit of U.S. Provisional Patent Application Ser. No. 62/816,136, filed Mar. 10, 2019, which is incorporated herein by reference in its entirety.

### FIELD OF THE INVENTION

The present invention generally relates to ocular drug or agent delivery devices, systems and methods, and ocular monitoring devices, systems and methods. More specifically, the present invention is directed to an ocular drug or agent delivery device, an ocular monitoring device, and methods thereof. The present invention is also for enhancing contact lens wearing comfort.

### BACKGROUND

Ocular drug or agent delivery devices, systems and methods, and ocular monitoring devices, systems and methods are known in the art. However, despite this, e many ophthalmic drug or agent delivery problems still exist today.

One problem of the many ophthalmic drug or agent delivery problems in the art is insufficient drug or agent amount to be delivered such that frequent applications of the drug or agent are required for sufficient relief of symptoms. More specifically, devices delivering drugs via dropper have problems which include but are not limited to
insufficient eye drop volume due to ocular volume (approximately 15 μL or microliter), no matter how many drops are added;
insufficient drug amount of to be delivered also due to the limit of eye drop osmolarity. More concentrated eye drops lead to hypertonicity, causing eye sting;
insufficient drug amount of to be delivered due to drug solubility. Insufficient drug amount cannot be loaded into the eyes;
the eye washes away drug-containing eye drops too quickly and efficiently, resulting in a higher than needed initial concentration to the eye when first applied and an insufficient concentration soon after application; and
the need for frequent and inconvenient application of eye drops, leading to problems due to contamination, such as toxicity, etc.

Another problem of the many ophthalmic drug or agent delivery devices in the art is the difficulty in achieving a sustained and controlled release rate of an ophthalmic drug formulations. Specifically,
drug release rate is not controlled in sustained manner, and is generally too fast when applied;
frequent tear flows and eye blinking, which both makes the sustained release more difficult; and
controlling an even concentration of the ophthalmic drug formulation is not achievable within a desired window.

The difficulty in maintaining a sustained release and even concentration of a drug formulation often necessitates frequent applications of the formulation to the eye, especially in treating conditions such as dry-eye. Such frequent application can result in various undesirable and harmful side effects, such as:
drug toxicity due to overdosing (systemic drug toxicity and side effects);
toxicity due to the preservatives added to the formulation, with the potential to cause systemic toxicity an serious ocular damage; and
interfering with natural eye function such as tear chemistry and tear film interruption.

Many ophthalmic drug or agent delivery systems in the art employ surfactants (and other various additives) in ophthalmic drug formulations to improve drug solubility and other properties. However, many surfactants often irritate the eyes.
A further problem of the many ophthalmic drug or agent delivery problems in the art is the lack of drug delivery methods toward a specific ocular area in the eye. Currently there are no ideal methods that deliver drugs toward a specific ocular area, and as a result, the drug is unselectively delivered to all parts of the eye, which may lead to side effects (e.g., toxicity) and wasting of drug.
An additional problem of the many ophthalmic drug or agent delivery devices known in the art is a poor drug delivery specifically to the cornea. Such delivery is extremely important for effective treatment to diseases or conditions specific to the cornea. However, there are currently no effective methods to deliver eye drops to the corneal area only. This problem is further complicated because the corneal area is sensitive to pain/sting which limits many medications or formulations that can be otherwise used, and the quantity of medication is further limited because any drug formulation with concentrations above isotonicity will cause more sting.

Additional problem of the many ophthalmic drug or agent delivery devices currently in the art is the use of ointments. While commonly available, the use of ointment formulations are generally tedious, are difficult to apply and may create discomfort in the patient. For example, an ointment formulation tends to get distributed everywhere in the eye area, typically making it difficult for a patient to see and read.

A further problem of the many ophthalmic drug or agent delivery devices in the art is the discomfort in using non-contact lens devices such as inserts. For example, using a non-contact lens device such as a bioerodible ocular device or inserts (see U.S. Pat. No. 5,366,739) can be uncomfortable, because the particle or carrier materials are inserted into sensitive area of the eyes, such as under the eyelids. Designing particles for ocular drug delivery in order to achieve the desired softness and dimensions is a complex process. Lack of softness and wettability and are primary the reasons such delivery devices can cause discomfort. To provide comfort, a material, such as gelatin (made from collagen hydrolysis) is commonly used. Although not necessarily cross-linked with harmful aldehydes, the particles, are commonly used by placement between a 2-lens jacket. The shape of the resulting device is unlike a contact lens shape which conforms to the eye, and as a result, can lead to discomfort. To provide improved wettability, certain bioerodible ocular devices require the polymeric materials to be hydrated first before insertion (see U.S. Pat. No. 4,865,846).

An additional problem of the many ophthalmic drug or agent delivery devices in the art is the material problems associated with bioerodible ocular devices. Bioerodable formulations, prepared by mixing a drug with a particle which is then suspended in a drug carrier and forms a temporary device within the eye. Although bioerodible ocular devices were intended to provide sustained release of drug formulations, many problems have hindered this technology to be accepted. In addition to the formulation being rapidly washed away by tears, some “bioerodible” polymers are hard to degrade in time which obviates their use in eye drops as drug media. Depending on the materials used, some bioerodible ocular devices may introduce unsafe materials into the eyes. For example, both the undegraded and degraded material may accumulate in the eye or enter into body, causing unwanted side effects. In addition, such devices may be difficult to remove, if necessary, during medication, which results in loss of control of the drug delivery profile. It may also be less safe for patient use, since it very difficult for a patient to determine if the materials are disintegrated completely and not be able to tell when the next dose should be applied, possibly leading to overdosing. Even if formulated in bioerodible form, the drug/ particle/carrier mixture can still retain some be toxicity for an indeterminate period.

Some ophthalmic drug or agent delivery devices known in the art are clear, water-miscible, liquid pharmaceutical vehicles and compositions which gel in situ at body temperature and are used for drug delivery to mucous membranes (see U.S. Pat. No. 4,100,271, developed by Cooper Laboratories, Inc., 1978). However such formulations are often hard to apply, may result in pain/stinging with many medications so formulations, may be unevenly distributed in the eyes, lack control in release location and release rate, and retention eye of the medication is not assured.

The present invention will improve, correct or solve at least one of the many ophthalmic drug or agent delivery problems in the art as disclosed above.

For eye analyte or biomarker monitoring or analysis, the current invention overcomes the low concentration problem by concentrating the analyte in the device and providing a sampling tool/method for next step analysis.

Further, the present invention overcomes one or more of the shortcomings of ocular drug or agent delivery devices, systems and methods, and ocular monitoring devices, systems and methods known in the art.

### SUMMARY OF THE INVENTION

The present invention is directed to an ocular drug or agent delivery device, ocular monitoring device, and to methods of treating a disease or adverse condition of the eye, said methods comprising delivering a drug to an eye of a patient in need thereof by applying a described drug delivery device.

Included in the invention is an ocular agent delivery device comprising a standard or modifying contact lens (1), wherein the rear surface (2) is treated with a medicament formulation, wherein

the medicament is selected from a liquid, an ointment, a film, a powder, a particle, a pre-gel, or gel, and

wherein the lens containing the medicament is optionally warmed by the user's figure tip for

a time sufficient to allow the liquid, an ointment, a film, a powder, a particle, a pre-gel, or gel to become uniformly distributed on the rear lens surface.

This embodiment is illustrated in FIG. 1.

Also included in the invention is an ocular agent delivery device comprising a plurality of layered contact lenses and independently selected medicament formulations, said medicament formulation selected from a liquid, an ointment, a film, a powder, a particle, a pre-gel, or gel,

in which a first lens (600) comprises an outer surface (620) and an inner surface

(610), the inner surface being treated with a layer of a first medicament formulation,

and covered concentrically with a second lens (640), and optionally to which are alternately added a second, third and fourth layer of medicament formulations and a third and fourth lens, such that the agent delivery device comprises from 1 to 4 lenses and from 1-4 medicament formulations.

This embodiment is illustrated in FIG. 6.

Also included in the invention is a two-lens ocular agent delivery device (100) comprising:

an inner lens (110) comprising a first section (120), a raised section (130), a front surface (140), a rear surface (142), and a side surface (144),

wherein said raised section (130) has a thickness that is greater than a thickness of said first section (120), and

wherein said inner lens (110) has a convex configuration when viewed from a side view going from said rear surface (142) toward said front surface (140); and

an outer lens (160) comprising an agent-containing section (170), an aperture (180), a front surface (190), a rear surface (192), and a side surface (194),

wherein said aperture (180) is dimensioned and configured for receiving said raised section (130) of said inner lens (110) when said inner lens (110) and said outer lens (160) are positioned proximate to one another during use,

wherein said inner lens (110) is positioned closer to an eyeball of an eye of a user than is said outer lens (160) to the eyeball during use,

wherein said outer lens (160) has a convex configuration when viewed from a side view going from said rear surface (192) toward the front surface (190) of said outer lens (160), and

wherein said agent-containing section (170) comprises at least one agent or drug (172) to effect at least one desirable effect to the eye or other body part, organ or tissue of the user U via the eye during use.

Also included in the present invention is a one-lens ocular agent delivery device (200) comprising:

a lens (210) comprising a first section (220), a recessed or receiving section (230), a front surface (240), a rear surface (242), and a side surface (244),

wherein said recessed or receiving section (230) has a thickness that is less than a thickness of said first section (220), and

wherein said lens (210) has a convex configuration when viewed from a side view going from said rear surface (242) toward said front surface (240); and

an agent-delivering or agent-containing insertion element (270) comprising a front surface (290), a rear surface (292), a side surface (194), and at least one agent or drug (272),

wherein said recessed or receiving section (230) of said lens (210) is dimensioned and configured for receiving said agent-delivering or agent-containing insertion element (270) when said lens (210) and said agent-delivering or agent-containing insertion element (270) are positioned proximate to one another during use, and

wherein said at least one agent or drug (272) produces at least one desirable effect to the eye or other body part, organ or tissue of the user U via the eye during use.

Other embodiments of the present invention improve, correct or solve at least one of the many ophthalmic agent delivery problems in the art disclosed and/or described and are described below and illustrated in the Drawings.

### BRIEF DESCRIPTION OF THE DRAWINGS

The accompanying figures, which are incorporated in and constitute a part of this specification, illustrate several aspects and together with the description serve to explain the principles of the invention.

FIG. 1 is a depiction of the standard contact lens, with two surfaces, and a typical method of handling the lens prior to application to the user's eye, using the user's fingertip.

FIG. 2 is an exploded side view of a first embodiment of an ocular agent delivery device according to the present invention, wherein the delivery device results from a first contact lens combining with a second contact lens containing a central aperture, as well as a view of the two lenses after coupled together.

FIG. 3A is a perspective and exploded view of the first embodiment of FIG. 2 of an ocular agent delivery device according to the present invention, wherein the delivery device results from a first contact lens combining with a second contact lens containing a central aperture.

FIG. 3B is a perspective view of the first embodiment of FIG. 2 of an ocular agent delivery device according to the present invention, wherein the delivery device results from a first contact lens combining with a second contact lens containing a central aperture, wherein

FIG. 3B is an assembled view of FIG. 3A and shows a view of the two lenses after being coupled together.

FIG. 4 is a exploded side perspective view of a single lens delivery device, showing a center insert of a drug or agent.

FIG. 5 is a front view of the ocular agent delivery device of FIG. 4.

FIG. 6 is an environmental front view of the ocular delivery device of FIG. 5, wherein the delivery device is positioned on an eyeball of an eye of a user, and wherein a blink from the eye of the user will help deliver an amount of an agent or drug to the eye.

FIG. 7 is a side and front view of an ocular agent delivery device wherein the delivery device has a first contact lens, an agent layer or film, and a second contact lens combining concentrically aligned with one another.

FIG. 8 is an exploded, side view of the ocular agent delivery device of FIG. 7.

FIG. 9 is a perspective view of the ocular agent delivery device of FIG. 7.

FIG. 10 is a schematic representation of four variations of the ocular agent delivery device of FIG. 7, each example differs in the permeability characteristics of the inner and outer lenses used.

FIG. 11 is a fourth embodiment of an ocular agent delivery device according to the present invention, wherein the delivery device results from a first contact lens combining with a second contact lens wherein the first lens contains a plurality of microneedles which are capable of penetrating the outer membrane layer of the cornea.

FIG. 12 is an environmental view of the device of FIG. 11.

FIG. 13 is a front view depiction of the inner (first) lens and the plurality of microneedles as they are arranged radially on the lens.

FIG. 14. is a side view of a two lens ocular agent delivery device in which the first lens (A) contains a plurality of wells into which a drug or agent may be placed, and where the first and second lens (B) are concentrically fitted together with a central locking pin in lens A and well in lens B.

FIG. 15 is an exploded, perspective view of the ocular agent delivery device of FIG. 14.

FIG. 16 is a front view of the first lens showing the wells and the insertion point for the second lens.

FIG. 17 is a front view of a variation of FIG. 16 in which the wells are not identical and where a drug or agent may be individually placed, and the insertion point for the second lens.

FIG. 18 is a front view of a second variation of FIG. 17 containing a plurality of rectangular keys, so as to provide unique address for each well and thereby controlling the specific drug or agent content of each well.

FIG. 19 is a perspective view of a fifth embodiment of an ocular agent delivery device according to the present invention, in which wherein the delivery device results from a first contact lens combining with a second contact lens and in which the second lens contains a plurality of wells into which a drug or agent may be placed and the first and second lens can be fitted together with a central locking pin and well.

FIG. 20 is a side view of the arrangement of a sixth embodiment of the invention in which a medicament formulation is distributed between two standard contact lenses.

It should be understood that the above-attached figures are not intended to limit the scope of the present invention in any way.

### DETAILED DESCRIPTION OF THE INVENTION

The present invention can be understood more readily by reference to the following detailed description of the invention and the Examples included therein.

Before the compositions, articles, systems, devices, and/or methods are disclosed and described, it is to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Embodiments of the present invention improve, correct or solve at least one of the many ophthalmic agent delivery problems in the art disclosed and/or described above by:
allowing larger drug loads to be applied;
allowing long-lasting drug loads to be applied;
release is controlled in several sustained ways;
application times can be reduced significantly, such as, but not limited to, once daily, once weekly, or once bi-weekly (which is the typical limit since that is the typical contact lens limit);
direct preservative exposure in sudden, large quantity is minimized;
ointment (instead of eye drops) use in the two-lens device (200) avoids the use of preservatives;
the two-lens device (200) greatly reduces preservative toxicity;
drug formulations with concentrated ingredients or water-free, or pure drug formulations that do not support microbial survival;
large molecule preservatives can be used, and they are restricted in two lenses for migration during use. Examples of such preservatives are polyquats and polylysine. Large molecule preservatives can be cross-linked to further reduce diffusion to ocular environments.

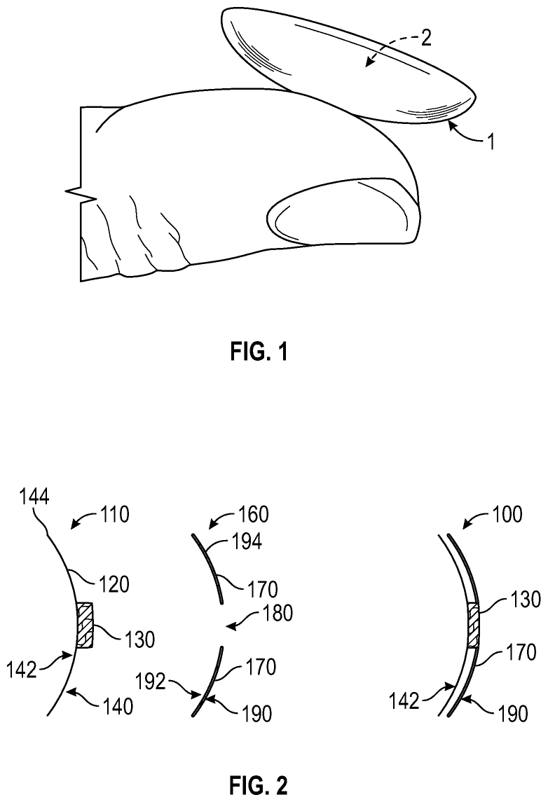

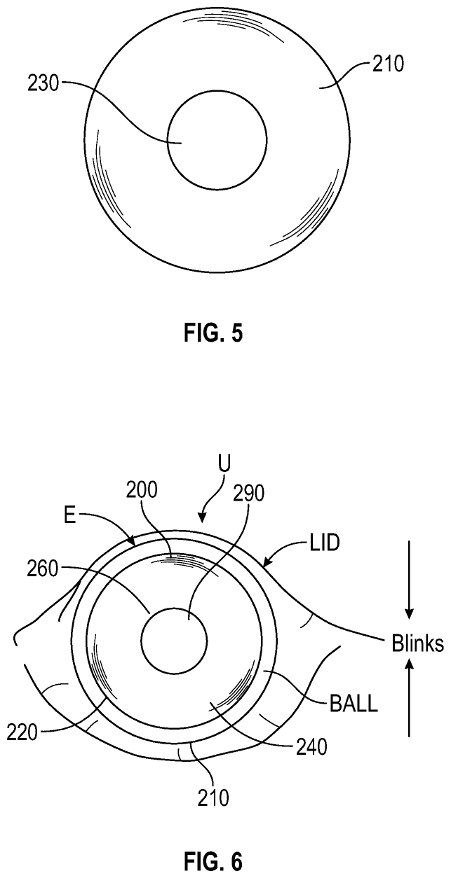

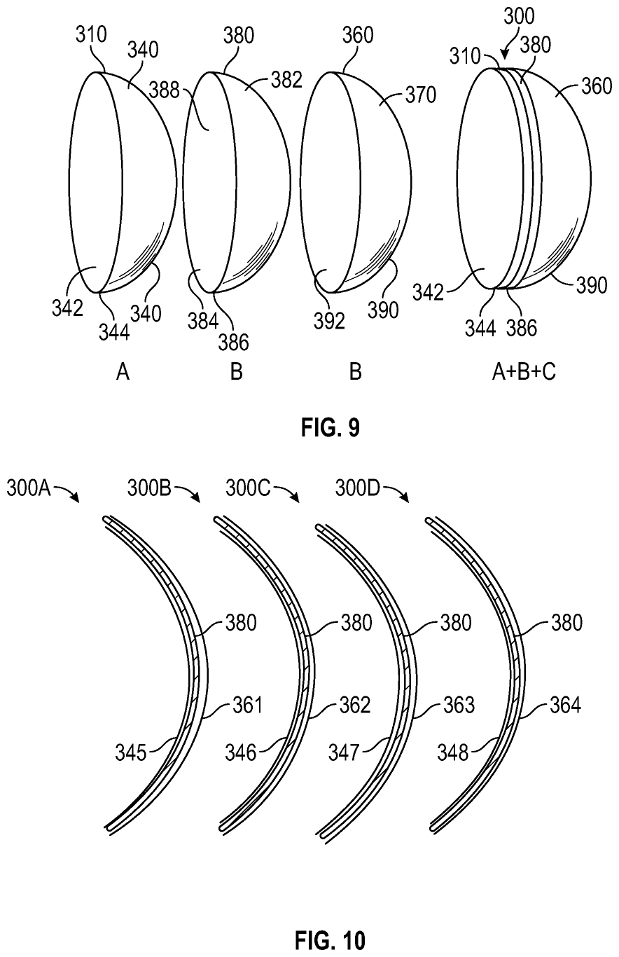

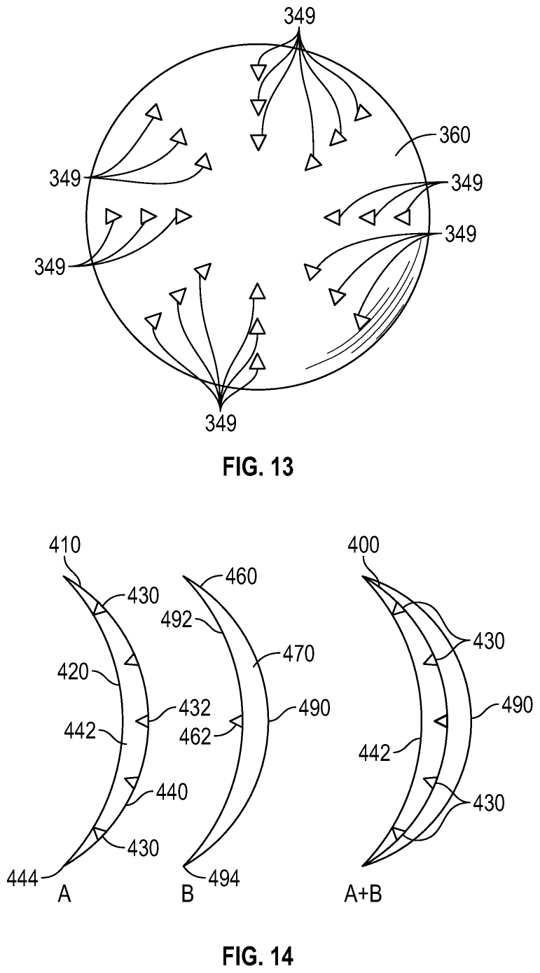

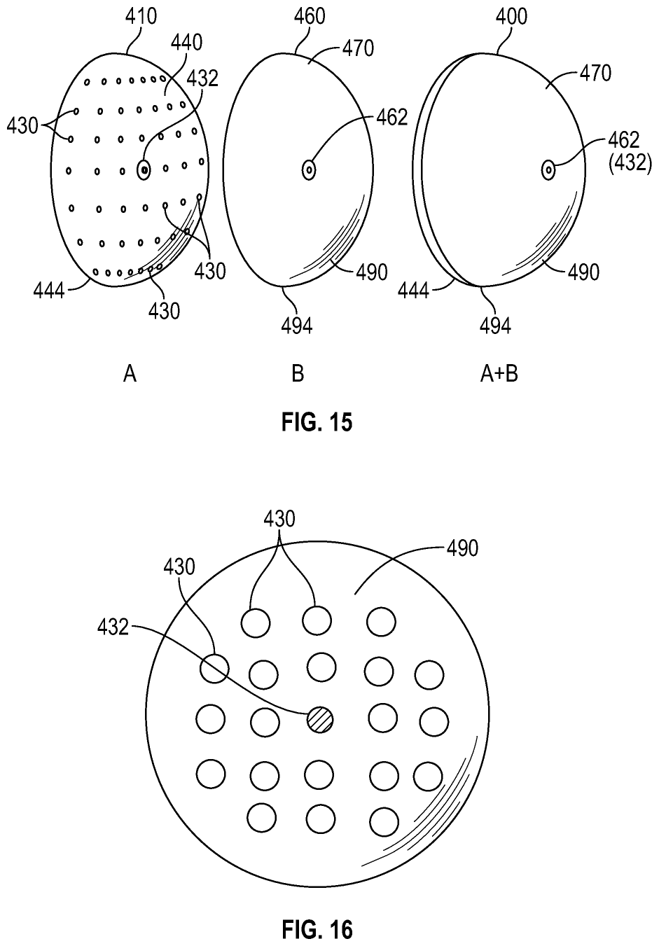

Referring to FIGS. 1-20 the present invention is directed to improved ocular agent delivery devices, improved ocular monitoring devices, and methods thereof. The present invention is also for enhancing contact lens wearing comfort.

The devices are normally applied to the eye by means well known in the art, typically by means of the fingertip of the user to whom the device is to be applied. (See FIG. 1). In addition to being a conventional and well-accepted means for applying lenses, this method also has the advantage that the drug or agent contained in the device becomes pre-warmed by contact with the human finger, which facilitates the solubilization and blending of the drug/agent formulations, especially when the formulation is in a gel or pre-gel state. The technology of in situ forming gels for delivery is known in the art, such as U.S. Pat. No. 5,318,780 and references therein.

As best shown in FIG. 1, a first embodiment of the present invention is an ocular agent delivery device comprising a standard contact lens (1), wherein the rear surface (2) is treated with a medicament formulation, wherein the medicament is selected from a liquid, an ointment, a pre-gel, or gel, and wherein the lens containing the medicament is optionally warmed by the user's figure tip for an ocular agent delivery device comprising a standard contact lens (1), wherein the rear surface (2) is treated with a medicament formulation, wherein the medicament is selected from a liquid, an ointment, a pre-gel, or gel, and wherein the lens containing the medicament is optionally warmed by the user's figure tip for a time sufficient to allow the ointment, liquid, pre-gel or gel to become uniformly distributed on the rear lens surface.

As best shown in FIG. 20, an second embodiment of the present invention is an ocular agent in which a first lens (600) comprises an outer surface (620) and an inner surface (610), the inner surface being treated with a layer of a first medicament formulation, and covered concentrically with a second lens (640), and optionally to which are alternately added a second, third and fourth layer of medicament formulations and a third and fourth lens, such that the agent delivery device comprises from 1 to 4 lenses and from 1-4 medicament formulations.

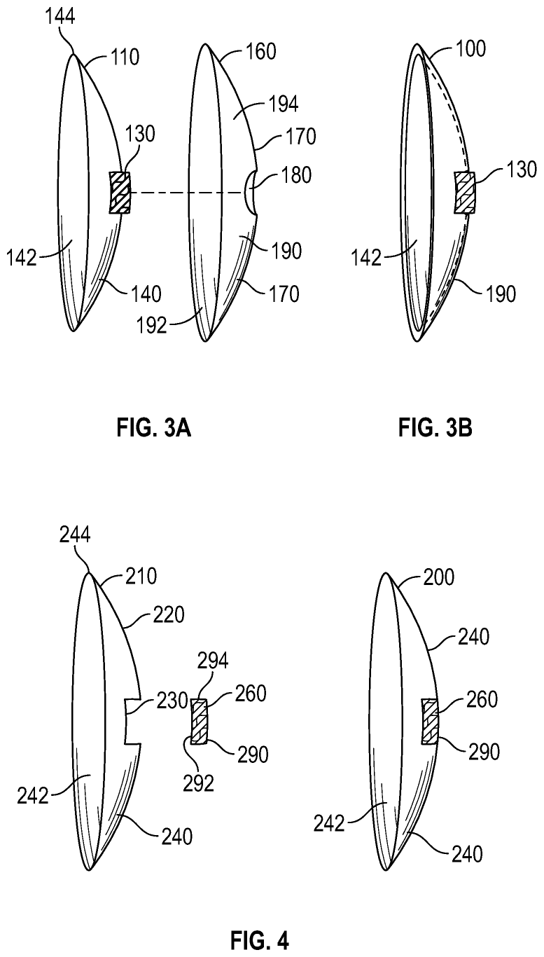

As best shown in FIGS. 2-3, a third embodiment of the present invention is a two-lens ocular agent delivery device (100) comprised of a first or inner lens (110) with a raised section (130), and a second or outer lens (160) with an agent-delivering or agent-containing section (170).

The first or inner lens (110) includes a first section (120), the raised section (130), a front surface (140), a rear surface (142), and a side surface (or, alternatively, a side edge) (144). Optionally, the first or inner lens (110) has a convex configuration when viewed from a side view going from the rear surface (142) toward the front surface (140) as shown in FIG. 2 such that the rear surface (142) is closer than the front surface (140) to an eyeball (BALL) of a user (U) during use of the two-lens ocular agent delivery device (100). Optionally, the first or inner lens (110) also has a generally circular configuration when viewing either of the front surface (140) or rear surface (142) as a whole themselves such that the generally circular configuration coincides with the configuration of the eyeball (BALL).

Optionally, the first section (120) is located at an outer or peripheral portion of the first or inner lens (110).

Optionally, the raised section (130) is located centrally within the first or inner lens (110), and has a thickness that is greater than the thickness of the first, outer or peripheral section (120).

The second or outer lens (160) includes the agent-delivering or agent-containing section (170), a cavity, hole or aperture (180), a front surface (190), a rear surface (192), and a side surface (or, alternatively, a side edge) (194). Optionally, the second or outer lens (160) has a convex configuration when viewed from a side view going from the rear surface (192) toward the front surface (190) as shown in FIG. 2 such that the rear surface (192) is closer than the front surface (190) to the first or inner lens (110) and eyeball (BALL of the user (U during use of the two-lens ocular agent delivery device (100). Optionally, the second or outer lens (160) also has a generally circular configuration when viewing either of the front surface (190) or rear surface (192) as a whole themselves such that the generally circular configuration coincides with the configuration of the eyeball (BALL).

Optionally, the agent-delivering or agent-containing section (170) is located at an outer or peripheral portion of the second or outer lens (160). The agent-delivering or agent-containing section (170) comprises at least one agent or drug (172) to be delivered to, to diffuse to, or to be contacted with a part or a component, such as an inner surface of an eyelid (LID), of an eye (E) of the user (U) to effect at least one desirable effect to the eye (E) and/or other body part, organ or tissue of the user (U) via the eye during use.

Optionally, the cavity, hole or aperture (180) is located centrally within the second or outer lens (160). The cavity, hole or aperture (180) is dimensioned and configured for accepting or receiving the raised section (130) of the first or inner lens (110) when the first or inner lens (110) and second or outer lens (160) are positioned proximate or secured to one another during use.

Optionally, the thickness of the raised section (130) of the first or inner lens (110) is about the same, or optionally, substantially the same, as the thickness of the agent-delivering or agent-containing section (170) of the second or outer lens (160) such that the raised section (130) and agent-delivering or agent-containing section (170) are flush or substantially flush with one another when the first or inner lens (110) and second or outer lens (160) are positioned proximate or secured to one another for use.

The inner lens (110) is positioned closer to the eyeball of the eye of the user than is the outer lens (160) to the eyeball during use.

As best shown in FIGS. 4-6, a fourth embodiment of the present invention is a one-lens ocular agent delivery device (200) comprised of a lens (210) with a recessed or receiving section (230), and an agent-delivering or agent-containing insertion element (290).

The lens (210) includes an outer or peripheral section (220), the recessed or receiving section (230), a front surface (240), a rear surface (242), and a side surface (or, alternatively, a side edge) (244). Optionally, the recessed or receiving section (230) is optionally located centrally within the lens (210). Optionally, the lens (210) has a convex configuration when viewed from a side view going from the rear surface (242) toward the front surface (240) as shown in FIG. 4 such that the rear surface (242) is closer than the front surface (240) to an eyeball (BALL of a user (U during use of the one-lens ocular agent delivery device (200). Optionally, the lens (210) also has a generally circular configuration when viewing either of the front surface (240) or rear surface (242) as a whole themselves such that the generally circular configuration coincides with the configuration of the eyeball (BALL).

The agent-delivering or agent-containing insertion element (260) includes a front surface (290), a rear surface (292), a side surface (294), and at least one agent or drug (292) to be delivered to, to diffuse to, or to be contacted with a part or a component, such as an inner surface of an eyelid (LID), of an eye (E) of the user (U) to effect at least one desirable effect to the eye (E) and/or other body part, organ or tissue of the user (U) via the eye.

Optionally, the recessed or receiving section (230) is located centrally within the lens (210). The recessed or receiving section (230) is dimensioned and configured for accepting or receiving the agent-delivering or agent-containing insertion element (260) when the lens (210 and agent-delivering or agent-containing insertion element (260) are positioned proximate or secured to one another for use.

Optionally, the thickness of some of the outer section (220) of the lens (210) is about the same, or optionally, substantially the same, as the thickness of the agent-delivering or agent-containing insertion element (260) such that the outer section (220) and agent-delivering or agent-containing insertion element (260) are flush or substantially flush with one another when the lens (210) and agent-delivering or agent-containing insertion element (260) are positioned proximate or secured to one another for use.

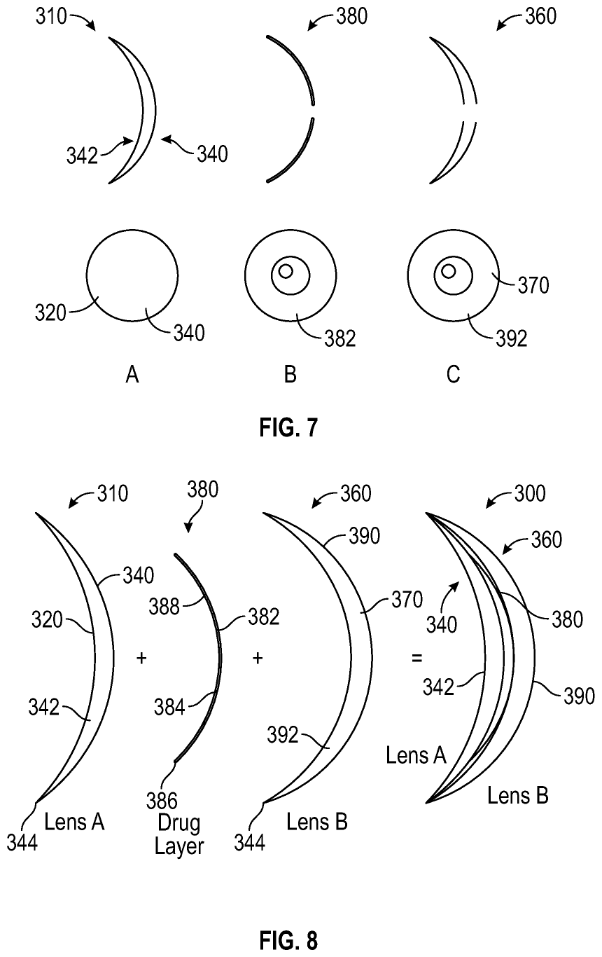

As best shown in FIGS. 7-13, a fifth embodiment of the present invention is a two-lens ocular agent delivery device (300) comprised of a first or inner lens (310, a second or outer cover lens 360), and at least one agent-delivering or agent-containing film or layer (380) positioned between the first or inner lens (310) and second or outer cover lens (360) during use.

The first or inner lens (310) includes a lens body (320), a front surface (340), a rear surface (342), and a side surface (or, alternatively, a side edge) (344). Optionally, the first or inner lens (310) has a convex configuration when viewed from a side view going from the rear surface (342) toward the front surface (340) as shown in FIG. 7 such that the rear surface (342) is closer than the front surface (340) to an eyeball (BALL) of a user (U) during use of the two-lens ocular agent delivery device (300). Optionally, the first or inner lens (310) also has a generally circular configuration when viewing either of the front surface (340) or rear surface (342) as a whole themselves such that the generally circular configuration coincides with the configuration of the eyeball (BALL).

The second or outer lens (360) includes a lens body (370), a front surface (390), a rear surface (392), and a side surface (or, alternatively, a side edge) (394). Optionally, the second or outer cover lens (360) has a convex configuration when viewed from a side view going from the rear surface (392) toward the front surface (390)as shown in FIG. 7 such that the rear surface (392) is closer than the front surface (390) to an eyeball (BALL of a user (U during use of the two-lens ocular agent delivery device (300). Optionally, the second or outer cover lens (360) also has a generally circular configuration when viewing either of the front surface (390) or rear surface (392) as a whole themselves such that the generally circular configuration coincides with the configuration of the eyeball (BALL. In one non-limiting example, the second or outer cover lens (360) is larger than the first or inner lens (310) such that the rear surface diameter and front surface diameter of the second or outer lens (360) are greater than the rear surface diameter and front surface diameter, respectively, of the first or inner lens (310). As another non-limiting example, the first or inner lens (310 and the second or outer cover lens (360) are substantially or exactly the same in size such that the rear surface diameter and front surface diameter of the second or outer lens (360 are substantially or exactly the same as the rear surface diameter and front surface diameter, respectively, of the first or inner lens (310). In another non-limiting example, the thickness of the lens body (320 of the first or inner lens (310) is greater than the thickness of the lens body (370) of the second or outer lens (360). As another non-limiting example, the thickness of the lens body 320 of the first or inner lens (310) is substantially or exactly the same as the thickness of the lens body (370) of the second or outer lens (360).

The agent-delivering or agent-containing film or layer (380) comprises a front surface (382), a rear surface (384), a side edge (386), and at least one agent or drug (388) to be delivered to or to diffuse to a part or a component, such as an inner surface of an eyelid (LID), of an eye (E) of the user (U) to effect at least one desirable effect to the eye E and/or other body part, organ or tissue of the user (U). Optionally, the agent-delivering or agent-containing film or layer (380) has a convex configuration when viewed from a side view going from the rear surface (384) toward the front surface (382) as shown in FIG. 7 such that such that the rear surface (384) is closer than the front surface (382) to an eyeball (BALL) of a user (U) during use of the two-lens ocular agent delivery device (300). Optionally, the agent-delivering or agent-containing film or layer (380) also has a generally circular configuration when viewing either of the front surface (382) or rear surface (384) as a whole themselves such that the generally circular configuration coincides with the configuration of the eyeball (BALL). Optionally, the agent-delivering or agent-containing film or layer (380) is dimensioned and configured to fit between the first or inner lens (310) and second or outer lens (360) when the first or inner lens (310) and second or outer lens (360) are positioned proximate or secured to one another for use such that the front surface (340) of the first or inner lens (310) is proximate to the rear surface (392) of the second or outer lens (360). Optionally, the agent-delivering or agent-containing film or layer (380) is also dimensioned and/or contain a sufficient amount of the at least one agent or drug (388) to be sufficiently effective in effecting at least one desirable effect to the eye E and/or other body part, organ or tissue of the user (U). This agent-delivering or agent-containing film or layer is manufactured by first preparing the medicament formulation followed by coating onto the carrier film or injection molding of the formulation/prepolymer mixture.

More specifically, the permeability of the inner lens A (310) and the outer lens C (360) can be selected in order provide controlled delivery of the drug or agent contained in the middle film or lens B (380), to specific regions of the eye. For example the regions can be the cornea, the edge (conjunctiva), the center (iris), or the entire eyeball. This is illustrated in FIG. 10, and summarized in Table 1 below.

TABLE 1

| | | | Rear | | |
|---|---|---|---|---|---|
| Config- | | Front | Lens | Rear | Eye |
| uration | Front | Lens | (Eyeball | Lens | Delivery |
| (380) | Lens | Permeable? | Side) | Permeable? | Region |
| A | (361) | Yes | (345) | No | Front/Iris |
| B | (362) | No | (346) | Yes | Cornea |
| C | (363) | No | (347) | No | Edge/ |
| | | | | | Conjunctiva |
| D | (364) | Yes | (348) | Yes | All Regions |

The permeability can be controlled by selection of the material that used in the manufacture of the lens. For example, polymers of methyl methacrylate (10-40%) or ethylene glycol monomethacrylate (20-40%) have a high permeability (increase in release rate of drug) compared to polymers of vinylpyrrolidone (10-50%), allyl methacrylate (0.1-5%) or ethylene dimethacrylate (01-5%). For example a lens material that would be well suited for the administration of pilocarpine hydrochloride or pilocarpine oil, would be one of low permeability since a very slow delivery rate is desired over an extended period for the treatment of glaucoma.

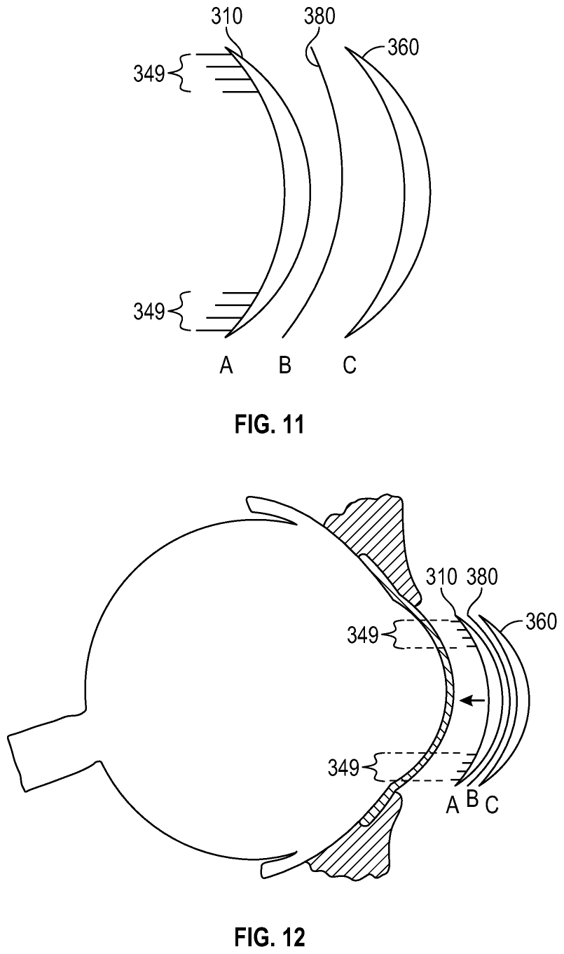

Another variation of this embodiment is illustrated in FIGS. 11-13. In this variation, the inner lens (360) closest to the eye is manufactured by molding of the hydrogel polymer lens with a plurality of micro needles (349), radially sited near the margin of the lens, such that when applied to the eye by the user, the lens adheres to the surface of the cornea and penetrates the thin outer layer of the eyeball. The penetrating action of the needles facilitate the delivery of drug or agent through the membrane of the eyeball and to the inner areas of the eyeball. By so doing, the delivery of drug or agent is expected to be more efficient, and can reduce the amount of drug required to be applied by the user. In is anticipated that a numbing or pain relieving agent can optionally be included in the drug or agent formulation lens/film (380), thus avoiding or minimizing any discomfort to the user.

As best shown in FIGS. 14-17, a sixth embodiment of the present invention is a two-lens ocular agent delivery device (400) comprised of a first or inner lens (410) with at least one agent well or reservoir (430, and a second or outer lens (460). Optionally, the delivery device (400) is further comprised of at least one agent or drug (450).

The first or inner lens (410) includes a lens body (420), the at least one agent well or reservoir (430), a first locking or securing element (432) of a locking mechanism of the delivery device (400), a front surface (440), a rear surface (442), and a side surface (or, alternatively, a side edge) (444). Optionally, the at least one agent well or reservoir (430) is a plurality of agent wells or reservoirs that are Optionally located at a plurality of predetermined locations along or about the front surface (440) of the first or inner lens (410). Each agent well or reservoir (430) is dimensioned to receive a predetermined amount or volume of at least one agent or a drug. Optionally, the first or inner lens (410) has a convex configuration when viewed from a side view going from the rear surface (442) toward the front surface (440) as shown in FIG. 14 such that the rear surface (442) is closer than the front surface (440) to an eyeball (BALL) of a user (U) during use of the two-lens ocular agent delivery device (400). Optionally, the first or inner lens (410) also has a generally circular configuration when viewing either of the front surface (440) or rear surface (442) as a whole themselves such that the generally circular configuration coincides with the configuration of the eyeball (BALL).

The second or outer lens (460) includes a lens body (470), a second locking or securing element (462) of the locking mechanism of the delivery device (400, a front surface (490), a rear surface (492), and a side surface (or, alternatively, a side edge) (494). The first locking or securing element (432 and second locking or securing element (462) form the locking mechanism of the delivery device (400) such that the first or inner lens (410) and second or outer lens (460) are able to be detachably locked or secured together prior to and during use. Optionally, the second or outer lens (460) has a convex configuration when viewed from a side view going from the rear surface (492) toward the front surface (490) as shown in FIG. 14 such that the rear surface (492) is closer than the front surface (490) to the first or inner lens (410) and eyeball (BALL) of the user (U) during use of the two-lens ocular agent delivery device (400). Optionally, the second or outer lens (460) also has a generally circular configuration when viewing either of the front surface (490) or rear surface (492) as a whole themselves such that the generally circular configuration coincides with the configuration of the eyeball (BALL).

The at least one agent or drug (450) is to be positioned within the at least one agent well or reservoir (430) such that it can be delivered to, to diffuse to, or to be contacted with a part or a component, such as an inner surface of an eyelid (LID), of an eye (E) of the user (U) to effect at least one desirable effect to the eye (E) and/or other body part, organ or tissue of the user (U).

A front view of this embodiment is illustrated in FIG. 16, showing the center locking well (432) of the inner lens (410) surrounded by a plurality of wells (430) to which are added the drug or agent.

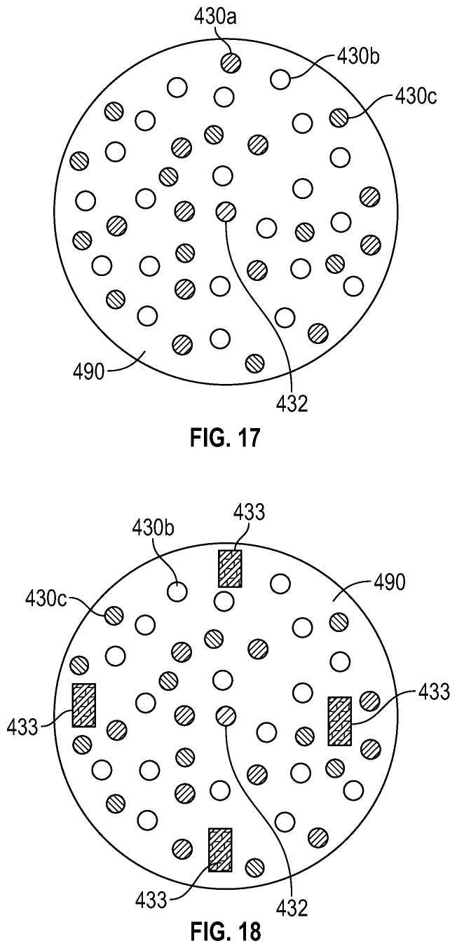

One variation of this embodiment is illustrated in FIG. 17, in which the individual wells (432) are configured to contain a plurality of drugs or agents, or drug or agent formulations. An example of the latter are drug or agent formulations with different time-release characteristics. Each well is programmed individually to contain a specific drug, agent or formulation of a drug or agent. One way to provide that each well has a unique address or specific location on the lens is the design of the lens as shown in FIG. 18. In this embodiment are included of one or more shaped locking keys in the surface of the lens, which hold the lens in position while the drug or agent is charged into the well. Is this example, the position of the lens is held in a specific orientation by rectangular shaped keys ho which are included in the pattern of the lens.

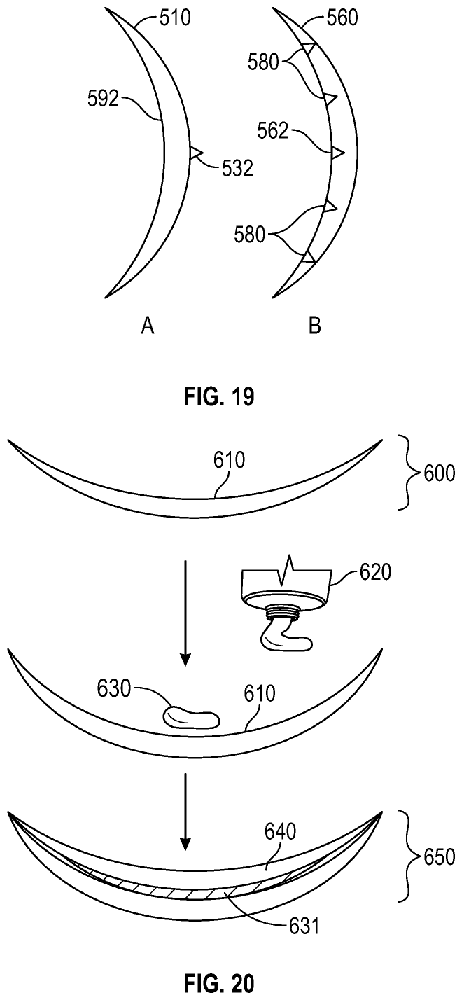

As best shown in FIG. 19, a sixth embodiment of the present invention is a two-lens ocular agent delivery device (500) comprised of a first or inner lens (510), and a second or outer lens (560) with at least one agent well or reservoir (580). Optionally, the delivery device (500) is further comprised of at least one agent or drug (550).

The first or inner lens (510) includes a lens body (520), a first locking or securing element (532) of a locking mechanism of the delivery device (500), a front surface (540), a rear surface (542), and a side surface (or, alternatively, a side edge) (544). Optionally, the first or inner lens (510) has a convex configuration when viewed from a side view going from the rear surface (542) toward the front surface (540) as shown in FIG. 19 such that the rear surface (542) is closer than the front surface (540) to an eyeball (BALL) of a user (U) during use of the two-lens ocular agent delivery device (500). Optionally, the first or inner lens (510) also has a generally circular configuration when viewing either of the front surface (540) or rear surface (542) as a whole themselves such that the generally circular configuration coincides with the configuration of the eyeball (BALL).

The second or outer lens (560) includes a lens body (570), the at least one agent well or reservoir ((580, a second locking or securing element (562) of the locking mechanism of the delivery device (500), a front surface (590), a rear surface (592), and a side surface (or, alternatively, a side edge) (594). Optionally, the at least one agent well or reservoir (580) is a plurality of agent wells or reservoirs that are Optionally located at a plurality of predetermined locations along or about the rear surface (592) of the second or outer lens (560). Each agent well or reservoir (580) is dimensioned to receive a predetermined amount or volume of an agent or a drug. The first locking or securing element (532) and second locking or securing element (562) form the locking mechanism of the delivery device (500) such that the first or inner lens (510) and second or outer lens (560) are able to be detachably locked or secured together prior to and during use. Optionally, the second or outer lens (560) has a convex configuration when viewed from a side view going from the rear surface (592) toward the front surface (590) as shown in FIG. 11 such that the rear surface (592) is closer than the front surface (590) to the first or inner lens (510) and eyeball (BALL) of the user (U) during use of the two-lens ocular agent delivery device (500). Optionally, the second or outer lens (560 also has a generally circular configuration when viewing either of the front surface (590 or rear surface (592) as a whole themselves such that the generally circular configuration coincides with the configuration of the eyeball (BALL).

The at least one agent or drug (550) is to be positioned within the at least one agent well or reservoir (580) such that it can be delivered to, to diffuse to, or to be contacted with a part or a component, such as an inner surface of an eyelid (LID, of an eye (E) of the user (U) to effect at least one desirable effect to the eye (E) and/or other body part, organ or tissue of the user (U).

An additional embodiment of the invention is the use of the devices for monitoring ocular conditions of the user. For example, the devices depicted in FIGS. 1-20, may be configured to have a monitoring agent composition within the lens or lenses. Such monitoring agents include imaging agents, diagnostic agents and the like. These include agents containing photoluminescent indicators and the like that allow the user to be regularly monitored by standard means for such conditions as glaucoma, macular degeneration, and the like.

Acronyms and Abbreviations

| | |
|---|---|
| AMD | acute macular degeneration |
| Ar-A | adenosine arabinoside |
| BALL | eyeball, cornea |
| C. | Celsius |
| CMC | carboxymethyl cellulose |
| EDTA | ethylenediaminetetraacetic acid |
| E | eye |
| IDU | 5-iodo-2′-deoxyuridine |
| LID | eyelid |
| poly I:C | polyinosinic:polycytidylic acid |
| PVOH | polyvinyl alcohol |
| PEG | polyethylene glycol |
| PEO | polyethylene glycol |
| U | user, patient |

The term “agent or drug,” “agent,” or “drug” as used in this application may be a drug; a medicine; a medicament; medicament formulation a pharmaceutical composition, formulation or solution. Such compositions include an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an antihypertensive, a muscle relaxant, and a tear modifying agent. Also included are beneficial agents that could be used as non-drugs to diagnose or relieve other adverse conditions of the eye, and include comfort compositions or diagnostic compositions.

The term “adverse conditions of eye” include dry eye, irritations or discomfort due to allergy, environmental conditions, or wearing of prescription contact lenses.

Examples of an agent or drug may be any agent or drug known to one of ordinary skill in the art that are used to effect at least one desirable effect to the eye and/or other body part, organ or tissue of the user via the eye. The agent or drug may be at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

The antibacterial/antimicrobial agent may be at least one member selected from the group consisting of tetracycline, sulfonamides, ampicillin trihydrate, oxytetracycline, penicillin, chloramphenicol, and nystatin.

Examples of antibacterial substances include beta-lactam antibiotics, such as cefoxitin, n-formamidoyl, thienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin, sulfonamides, aminoglycoside antibiotics, such as gentamycin, kanamycin, amikacin, sisomicin and tobramycin, nalidixic acid and its analogs such as norfloxacin and antimicrobial combinations including fluoroalanine/pentizidone, nitrofurazones and analogs thereof.

Examples of antihistamines and decongestants include pyrilamine, chlorpheniramine, tetrahydrozoline, antazoline and analogs thereof, and mast-cell inhibitors of histamine release such as cromolyn.

Examples of antiparasitic agents and/or anti-protozoal compounds such as ivermectin, pyrimethamine, trisulfapidimidine, clindamycin and corticosteroid preparations.

Examples of antifungal agents include amphotericin B, nystatin, tlucytosine, natamycin and miconazole.

Examples of antiviral agents include cyclovir, 5-iodo-2′-deoxyuridine (IDU), adenosine arabinoside (Ara-A), trifluorothymidine, interferon, and interferon-inducing agents such as poly I:C (Polyinosinic:polycytidylic acid).

Examples of a mydriatic agent include atrophine, homatropine, scopolamine, hydroxyamphetamine, ephedrine, cocaine, tropicamide, phenylephrine, cyclopentolate, oxyphenonium, eucatropine, and analogs thereof.

Examples of miotics and anticholinergic agents include chothiophate, pilocarpine, physostigmine salicylate, diisopropylfluorophosphate, epinephrine, dipivaloylepinephrine, neostigmine, echothiopate iodide, demecarium bromide, carbamoyl choline chloride, methacholine, bethanechol, and analogs thereof.

Ophthalmic agents include other agents which may be used alone or in combination with other bioactive agents above, such as antibiotic/anti-inflammatory combinations such as the combination of neomycin sulfate and dexamethasone sodium phosphate, and combinations concomitantly used for treating glaucoma, for example, a combination of timolol maleate and aceclidine.

Examples of other ophthalmic agents include one or more tear modifying agents; agents used as an adjuvant in surgery, such as alpha-chymotrypsin and hyaluronidase; chelating agents such as ethylenediaminetetraacetic acid (EDTA) and deferoxamine; immunosuppressants and anti-metabolites such as methotrexate, cyclophosphamide, 6-mercapto methotrexate, cyclophosphamide, 6-mercaptopurine and azathioprine. Medicaments available to treat dry eye include Restasis ®, Hyalein® and Diquas®.

Tear modifying agents include for example, a calcium compound (such as one that includes at least one of CaCO₃, CMC, PVOH, vitamin A, a vitamin A precursor, a lubricating substance, a retinoid, a vitamin A alcohol, a beta blocker, and a carbonic anhydrase inhibitor) and a lipid compound.

The agent or drug may be a sustained-release type or a controlled-release type. The controlled-release type may be an agent rate-controlling formulation of said agent that provides a controlled release of said agent.

The agent or drug may be loaded into or onto a carrier. The carrier, or vehicle, which facilitates the delivery and/or distribution of the drug, may be at least one member selected from the group consisting of silica, diatomaceous earth, starch, PEG, PEO, a synthetic polymer, and a natural polymer.

Medicaments available to treat acute macular degeneration (AMD) include Lucentis®, Visudyne®, Avastin®, Eylea®, and Macugen®.

Available anti-allergy or anti-inflammatory drugs include cortisone, hydrocortisone, hydrocortisone acetate, betamethasone, dexamethasone, dexamethasone sodium phosphate, preunisone, methylprednisolone, medrysone, fluorometholone, prednisolone, preunisolone sodium phosphate, triamcinolone, indomethacin, sulindac, its salts and its corresponding sulfides, and analogs thereof. Commercial products include Patanol®, Pataday®, TobraDex®, Vigamox®, Bepreve®, Xibrom®, and Moxeza®.

Examples of anti-glaucoma agents includetimolol, and especially its maleic salt and R-timolol and a combination of timolol or R-timolol with pilocarpine, as well as many other adrenergic agonists and/or antagonists: epinephrine and an epinephrine complex, or prodrugs such as bitartrate, borate, hydrochloride and dipivefrine derivatives; carbonic anhydrase inhibitors such as acetazolamide, dichlorphenamide, 2-(p-hydroxyphenyl)-thiophenesulfonamide, 6-hydroxy-2-benzothiazolesulfonamide, and 6-pivaloyloxy-2-benzothiazolesulfonamide. Commercial products include Xalatan®/Xalacom®, Cosopt®/Trusopt®, Travatan®/Travatan Z®/DuoTrav®, Lumigan®, Latisse®, Ganfort® (Lumigan® +Tumolol®), Alphagan® /Combigan®, and Azopt®.

As non-limiting examples, the agent may be made in different formulations, in different forms such as hydrogels, pre-gels, or other gel medication, powders, salts, liquid or solid formulation etc.

The drug can be formulated in a pre-polymer solution, i.e., is a in liquid status and becomes gel upon warming. In some cases it would be advantageous to formulate the drug/pre-polymer so as to control the temperature of gelation to be below 37° C. For example, the drug/pre-polymer solution can be kept refrigerated and added to the lens to be treated (e.g., lens surface (2) of FIG. 1, or lens surface (610) of FIG. 20) just prior to the insertion to the eye by the user. The warming to rt or by contact with the user's finger results in formation or partial formation of the gel, the higher viscosity keeping the drug formulation on the surface of the lens long enough to apply without spilling. Once applied to the lens and inserted into the eye, the gel can further congeal to better conform to the eye shape of the individual user.

It is to be understood that the present invention is not limited to the embodiments described above or as shown in the attached figures, but encompasses any and all embodiments within the spirit of the invention.

What is claimed is:

1. An ocular agent delivery device comprising a contact lens (1), wherein the rear surface (2) is treated with a medicament formulation, wherein
the medicament is selected from a liquid, an ointment, a film, a powder, a particle, a pre-gel, or gel, and
wherein the lens containing the medicament is optionally warmed by the user's figure tip for
a time sufficient to allow the liquid, an ointment, a film, a powder, a particle, a pre-gel, or gel to become uniformly distributed on the rear lens surface.

2. An ocular agent delivery device comprising a plurality of layered contact lenses and independently selected medicament formulations, said medicament formulation selected from an ointment, liquid, pre-gel or gel,
in which a first lens (600) comprises an outer surface (620) and an inner surface (610), the inner surface being treated with a layer of a first medicament formulation, and covered concentrically with a second lens (640), and
optionally to which are alternately added a second, third and fourth layer of medicament formulations and a third and fourth lens, such that the agent delivery device comprises from 1 to 4 lenses and from 1-4 medicament formulations.

3. The two-lens ocular agent delivery device (100) of claim 2, in which comprising:
the first lens (110) comprises a first section (120), a raised section (130), a front surface (140), a rear surface (142), and a side surface (144),
wherein said raised section (130) has a thickness that is greater than a thickness of said first section (120), and
wherein said first lens (110) has a convex configuration when viewed from a side view going from said rear surface (142) toward said front surface (140); and wherein
the second outer lens (160) further comprises an agent-containing section (170), an aperture (180), a front surface (190), a rear surface (192), and a side surface (194),
wherein said aperture (180) is dimensioned and configured for receiving said raised section (130) of said inner lens (110) when said inner lens (110) and said outer lens (160) are positioned proximate to one another during use,
wherein said inner lens (110) is positioned closer to an eyeball of an eye of a user than is said outer lens (160) to the eyeball during use,
wherein said outer lens (160) has a convex configuration when viewed from a side view going from said rear surface (192) toward the front surface (190) of said outer lens (160), and
wherein said agent-containing section (170) comprises at least one agent or drug (172) to effect at least one desirable effect to the eye or other body part, organ or tissue of the user U via the eye during use.

4. The ocular agent delivery device (100) according to claim 3, wherein said raised section (130) is located centrally within said inner lens (110), and wherein said aperture (180) is located centrally within said outer lens (160).

5. The ocular agent delivery device according to claim 1, wherein said at least one agent or drug is at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

6. The ocular agent delivery device according to claim 5, wherein said antibacterial/antimicrobial agent is at least one member selected from the group consisting of tetracycline, sulfonamides, ampicillin trihydrate, oxytetracycline, penicillin, chloramphenicol, and nystatin.

7. The ocular agent delivery device according to claim 5, wherein said tear modifying agent is at least one member selected from the group consisting of a calcium compound and a lipid compound.

8. The ocular agent delivery device according to claim 1, wherein said agent or drug is selected from a sustained-release type and a controlled-release type.

9. The ocular agent delivery device according to claim 1, further comprising a carrier, wherein said medicament is loaded into or onto said carrier.

10. The ocular agent delivery device according to claim 9, wherein said carrier is at least one member selected from the group consisting of silica, diatomaceous earth, starch, PEG, PEO, a synthetic polymer, and a natural polymer.

11. A one-lens ocular agent delivery device (200) comprising:
a lens (210) comprising a first section (220), a recessed or receiving section (230), a front surface (240), a rear surface (242), and a side surface (244),
wherein said recessed or receiving section (230) has a thickness that is less than a thickness of said first section (220), and
wherein said lens (210) has a convex configuration when viewed from a side view going from said rear surface (242) toward said front surface (240); and
an agent-delivering or agent-containing insertion element (270) comprising a front surface (290), a rear surface (292), a side surface (194), and at least one agent or drug (272),
wherein said recessed or receiving section (230) of said lens (210) is dimensioned and configured for receiving said agent-delivering or agent-containing insertion element (270) when said lens (210) and said agent-delivering or agent-containing insertion element (270) are positioned proximate to one another during use, and
wherein said at least one agent or drug (272) produces at least one desirable effect to the eye or other body part, organ or tissue of the user U via the eye during use.

12. The ocular agent delivery device (200) according to claim 11, wherein said recessed or receiving section (230) is located centrally within said lens (210).

13. The ocular agent delivery device (200) according to claim 11, wherein said at least one agent or drug (272) is at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

14. The ocular agent delivery device (200) according to claim 13, wherein said antibacterial/antimicrobial agent is at least one member selected from the group consisting of tetracycline, sulfonamides, ampicillin trihydrate, oxytetracycline, penicillin, chloramphenicol, and nystatin.

15. The ocular agent delivery device (200) according to claim 13, wherein said tear modifying agent is at least one member selected from the group consisting of a calcium compound and a lipid compound.

16. The ocular agent delivery device (200) according to claim 11, wherein said agent or drug (272) is selected from a sustained-release type and a controlled-release type.

17. The ocular agent delivery device (200) according to claim 11, further comprising a carrier, wherein said agent or drug (272) is loaded into or onto said carrier.

18. The ocular agent delivery device (200) according to claim 17, wherein said carrier is at least one member selected from the group consisting of silica, diatomaceous earth, starch, PEG, PEO, a synthetic polymer, and a natural polymer.

19. A two-lens ocular agent delivery device (300) comprising:
an inner lens (310) comprising a lens body (320), a front surface (340), a rear surface (342), and a side surface (344),
wherein said inner lens (310) has a convex configuration when viewed from a side view going from said rear surface (342) toward said front surface (340);
an outer lens (360) comprising a lens body (370), a front surface (390), a rear surface (392), and a side surface (394),
wherein said outer lens (360) has a convex configuration when viewed from a side view going from said rear surface (392) toward said front surface (390) of said outer lens (360), and
wherein said inner lens (310) is positioned closer to an eyeball of an eye of a user than is said outer lens (360) to the eyeball during use; and
at least one agent-delivering or agent-containing film or layer (380) comprising a front surface (382), a rear surface (384), a side edge (386), and at least one agent or drug (372).
wherein said at least one agent-delivering or agent-containing film or layer (380) is dimensioned and configured to fit between said inner lens (310) and said outer lens (360) when said inner lens (310) and said outer lens (360) are positioned proximate or secured to one another for use such that said front surface (340) of said inner lens (310) is proximate to said rear surface (392) of said outer lens (360) positioned between said inner lens (310) and said outer lens (360) during use,
wherein said at least one agent-delivering or agent-containing film or layer (380) has a convex configuration when viewed from a side view going from said rear surface (384) toward said front surface (382), and
wherein said at least one agent or drug (372) effects at least one desirable effect to the eye or other body part, organ or tissue of the user U via the eye during use.

20. The ocular agent delivery device (300) according to claim 19, wherein said at least one agent or drug (372) is at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

21. The ocular agent delivery device (300) according to claim 20, wherein said antibacterial/antimicrobial agent is at least one member selected from the group consisting of tetracycline, sulfonamides, ampicillin trihydrate, oxytetracycline, penicillin, chloramphenicol, and nystatin.

22. The ocular agent delivery device (300) according to claim 20, wherein said tear modifying agent is at least one member selected from the group consisting of a calcium compound and a lipid compound.

23. The ocular agent delivery device (300) according to claim 19, wherein said agent or drug (372) is selected from a sustained-release type and a controlled-release type.

24. The ocular agent delivery device (300) according to claim 23, wherein said controlled-release type is an agent rate-controlling formulation of said agent that provides a controlled release of said agent.

25. The ocular agent delivery device (300) according to claim 19, further comprising a carrier, wherein said agent or drug (372) is loaded into or onto said carrier.

26. The ocular agent delivery device (300) according to claim 25, wherein said carrier is at least one member selected from the group consisting of silica, diatomaceous earth, starch, PEG, PEO, a synthetic polymer, and a natural polymer.

27. The ocular agent delivery device (300) according to claim 19, wherein said outer lens (360) is larger than said inner lens (310) such that a rear surface diameter and a front surface diameter of said outer lens (360) are greater than a rear surface diameter and a front surface diameter, respectively, of said inner lens (310).

28. The ocular agent delivery device (300) according to claim 19, wherein a thickness of said lens body (320) of said inner lens (310) is greater than a thickness of said lens body (370) of said outer lens (360).

29. A two-lens ocular agent delivery device (400) comprising:
an inner lens (410) comprising a lens body (420), a front surface (440), a rear surface (442), a side surface (444), at least one agent well or reservoir (430), and a first locking or securing element (432) of a locking mechanism of said ocular agent delivery device (400),
wherein said inner lens (410) has a convex configuration when viewed from a side view going from said rear surface (442) toward said front surface (440), and
wherein said at least one agent well or reservoir (430) is dimensioned to receive a predetermined amount or volume of at least one agent or a drug; and
an outer lens (460) comprising a lens body (470), a front surface (490), a rear surface (492), a side surface (494), and a second locking or securing element (462) of said locking mechanism of said ocular agent delivery device (400),
wherein said outer lens (460) has a convex configuration when viewed from a side view going from said rear surface (492) toward said front surface (490) of said outer lens (460), and
wherein said inner lens (410) is positioned closer to an eyeball of an eye of a user than is said outer lens (460) to the eyeball during use.

30. The ocular agent delivery device (400) according to claim 29, further comprising at least one agent or drug (472).

31. The ocular agent delivery device (400) according to claim 30, wherein said at least one agent or drug (472) is at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

32. The ocular agent delivery device (400) according to claim 31, wherein said antibacterial/antimicrobial agent is at least one member selected from the group consisting of tetracycline, sulfonamides, ampicillin trihydrate, oxytetracycline, penicillin, chloramphenicol, and nystatin.

33. The ocular agent delivery device (400) according to claim 31, wherein said tear modifying agent is at least one member selected from the group consisting of a calcium compound and a lipid compound.

34. The ocular agent delivery device (400) according to claim 31, wherein said agent or drug 172 is selected from a sustained-release type and a controlled-release type.

35. The ocular agent delivery device (400) according to claim 34, wherein said controlled-release type is an agent rate-controlling formulation of said agent that provides a controlled release of said agent.

36. The ocular agent delivery device (400) according to claim 31, further comprising a carrier, wherein said agent or drug (472) is loaded into or onto said carrier.

37. The ocular agent delivery device (400) according to claim 36, wherein said carrier is at least one member selected from the group consisting of silica, diatomaceous earth, starch, PEG, PEO, a synthetic polymer, and a natural polymer.

38. A two-lens ocular agent delivery device (500) comprising:
an inner lens (510) comprising a lens body (520), a front surface (540), a rear surface (542), a side surface (544), and a first locking or securing element (532) of said locking mechanism of said ocular agent delivery device (500),
wherein said inner lens (510) has a convex configuration when viewed from a side view going from said rear surface (542) toward said front surface (540); and
an outer lens (560) comprising a lens body (570, a front surface (590), a rear surface (592), a side surface (594), at least one agent well or reservoir (580),
wherein said well or reservoir (580) is dimensioned to receive a predetermined amount or volume of at least one agent or a drug,
and a second locking or securing element (562) of said locking mechanism of said ocular agent delivery device (500),
wherein said outer lens (560) has a convex configuration when viewed from a side view going from said rear surface (592) toward said front surface (590) of said outer lens (560), and
wherein said at least one agent well or reservoir (580) is dimensioned to receive a predetermined amount or volume of at least one agent or a drug, and
wherein said inner lens (510) is positioned closer to an eyeball of an eye of a user than is said outer lens (560) to the eyeball during use.

39. The ocular agent delivery device (500) according to claim 38, wherein said inner lens (510) has a generally circular configuration when viewing either of said front surface (540) or said rear surface (542) as a whole themselves.

40. The ocular agent delivery device (500) according to claim 38, wherein said outer lens (560) has a generally circular configuration when viewing either of said front surface (590) or said rear surface (592) as a whole themselves.

41. The ocular agent delivery device (500) according to claim 38, further comprising at least one agent or drug (550).

42. The ocular agent delivery device (500) according to claim 41, wherein said at least one agent or drug (550) is at least one member selected from the group consisting of an antibacterial/antimicrobial agent, an antihistamine agent, a decongestant agent , an anti-inflammatory, an antiparasitic, a miotic, an anticholinergic, an antiviral, a local anesthetic, an antifungal, an amoebicidal, a trichomonocidal, an analgesic, a mydriatic, an antiglaucoma drug, a carbonic anhydrase inhibitor, an ophthalmic agent, an ophthalmic agent used as an adjuvant in surgery, a chelating agent, an antineoplastic, an antihypertensive, a muscle relaxant, and a tear modifying agent.

43. The ocular agent delivery device (500) according to claim 42, wherein said antibacterial/antimicrobial agent is at least one member selected from the group consisting of tetracycline, sulfonamides, ampicillin trihydrate, oxytetracycline, penicillin, chloramphenicol, and nystatin.

44. The ocular agent delivery device (500) according to claim 42, wherein said tear modifying agent is at least one member selected from the group consisting of a calcium compound and a lipid compound.

45. The ocular agent delivery device (500) according to claim 41, wherein said agent or drug (550) is selected from a sustained-release type and a controlled-release type.

46. The ocular agent delivery device (500 according to claim 45, wherein said controlled-release type is an agent rate-controlling formulation of said agent that provides a controlled release of said agent.

47. The ocular agent delivery device (500) according to claim 38, further comprising a carrier, wherein said agent or drug (550) is loaded into or onto said carrier.

48. The ocular agent delivery device (500) according to claim 47, wherein said carrier is at least one member selected from the group consisting of silica, diatomaceous earth, starch, PEG, PEO, a synthetic polymer, and a natural polymer.

49. A method of treating a disease or adverse condition of the eye, said method comprising delivering a drug to an eye of a patient in need thereof, by applying the device of claim 1 to the eye, said device containing the drug on the raised section (130).

50. A method of treating a disease or adverse condition of the eye, said method comprising delivering a drug to an eye of a patient in need thereof, by applying the device (650) of claim 2 to the eye, said device containing the drug or medicament on the agent-containing insertion element section.

51. A method of treating a disease or adverse condition of the eye, said method comprising delivering a drug to an eye of a patient in need thereof, by applying the device (300) of claim 20 to the eye, said device containing the drug or medicament on at least one agent-delivering or agent-containing film or layer (380).

52. A method of treating a disease or adverse condition of the eye, said method comprising delivering a drug to an eye of a patient in need thereof, by applying the device (400) of claim 30 to the eye, said device containing the drug or medicament in at least one agent well or reservoir (430).
